(19) United States
(12) Patent Application Publication
(10) Pub. No.: US 2011/0185796 A1
(43) Pub. Date: Aug. 4, 2011

(54) CISATRACURIUM DERIVATIVES, PREPARATION AND USES THEREOF

(75) Inventors: Oded Arad, Rehovot (IL); Vladimir Naddaka, Petach Tikva (IL); Eyal Klopfer, Tel Aviv (IL); Shady Saeed, Haifa (IL); Lior Shahar, Kyriat Ono (IL); Vitaly Shteinman, Tel Aviv (IL)

(73) Assignee: CHEMAGIS Ltd., Bnei Brak (IL)

(21) Appl. No.: 12/990,428

(22) PCT Filed: Apr. 28, 2009
(86) PCT No.: PCT/IL09/00452

§ 371 Date: Nov. 16, 2010

Related U.S. Application Data
(60) Provisional application No. 61/049,620, filed on May 1, 2008.

(51) Int. Cl. G01N 30/84; C07D 217/02

(52) U.S. Cl. 73/61.55; 546/150

(57) ABSTRACT

The present invention provides compounds which are useful, e.g., as reference markers for analyzing the purity of cisatracurium and salts thereof, a test method for determining the said purity and processes for preparing reference markers.

### TECHNICAL FIELD

The present invention relates to compounds which are useful, e.g., as reference markers for analyzing the purity of cisatracurium and salts thereof, and to the preparation of such compounds.

### BACKGROUND OF THE INVENTION

Cisatracurium besylate has the chemical name (1R,1′R,2R,2′R)-2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methyl-isoquinolinium dibenzenesulfonate and is represented by the structural formula (I) below:

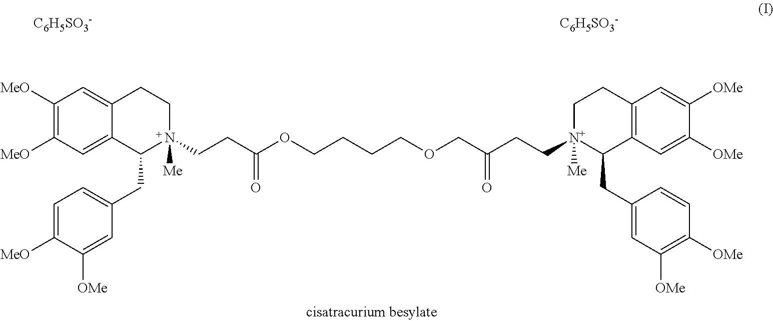

Cisatracurium besylate is the dibenzenesulfonate salt of 1R-cis,1′R-cis isomer of atracurium. The atracurium compound has four chiral centers resulting in 16 possible isomers. Due to the symmetry of the molecule, the number of isomers is reduced to 10. The possible isomers of atracurium are detailed by J. B. Stenlake et al. in “Biodegradable neuromuscular blocking agents,” Eur. J. Med. Chem.—Chem. Ther., vol. 19, issue 5, pp. 441-450 (1984).

Cisatracurium besylate is a nondepolarizing neuromuscular blocking agent indicated for inpatients and outpatients as an adjunct to general anesthesia, to facilitate tracheal intubation, and to provide skeletal muscle relaxation during surgery or mechanical ventilation in the Intensive Care Unit (ICU). Cisatracurium besylate possesses an activity that is superior to atracurium besylate, with significantly less side effects.

Cisatracurium besylate is marketed in the United States and Europe by Glaxo and Abbott Laboratories under the trade name Nimbex®. Nimbex® is a sterile, non-pyrogenic aqueous solution that is adjusted to pH 3.25 to 3.65 with benzenesulfonic acid. The drug is provided in 2.5 ml, 5 ml and 10 ml ampoules having strength of 2 mg/ml cisatracurium besylate. In addition, a 30 ml vial containing 5 mg/ml cisatracurium besylate is also available.

Cisatracurium besylate slowly loses potency with time a rate of approximately 5% per year under refrigeration (5° C.). Nimbex should be refrigerated at 2° to 8° C. (36° to 46° F.) in the carton to preserve potency. The rate of loss in potency increases to approximately 5% per month at 25° C. (77° F.).

Atracurium besylate, otherwise known as 2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methyl-isoquinolinium dibenzenesulfonate, was first disclosed in U.S. Pat. No. 4,179,507 (hereinafter U.S. '507). U.S. '507 describes a series of bis veratryl isoquinolinium quaternary ammonium salts, preferably among them is atracurium besylate. The synthesis of atracurium besylate, as taught in U.S. '507, involves the coupling of (±)-tetrahydropapaverine base (compound II), with 1,5-pentamethylene diacrylate (compound III). Treatment of the resulting tertiary amine base with oxalic acid results in the isolation of N,N′-4,10-dioxa-3,11-dioxotridecylene-1,13-bis-tetrahydropapaverine dioxalate (compound IV). The dioxalate salt (compound IV) is converted to the free base (compound V), which is treated with methyl benzenesulfonate. The resulting product, atracurium besylate (compound VI), is precipitated and isolated. Scheme 1 below illustrates the chemical pathway described above.

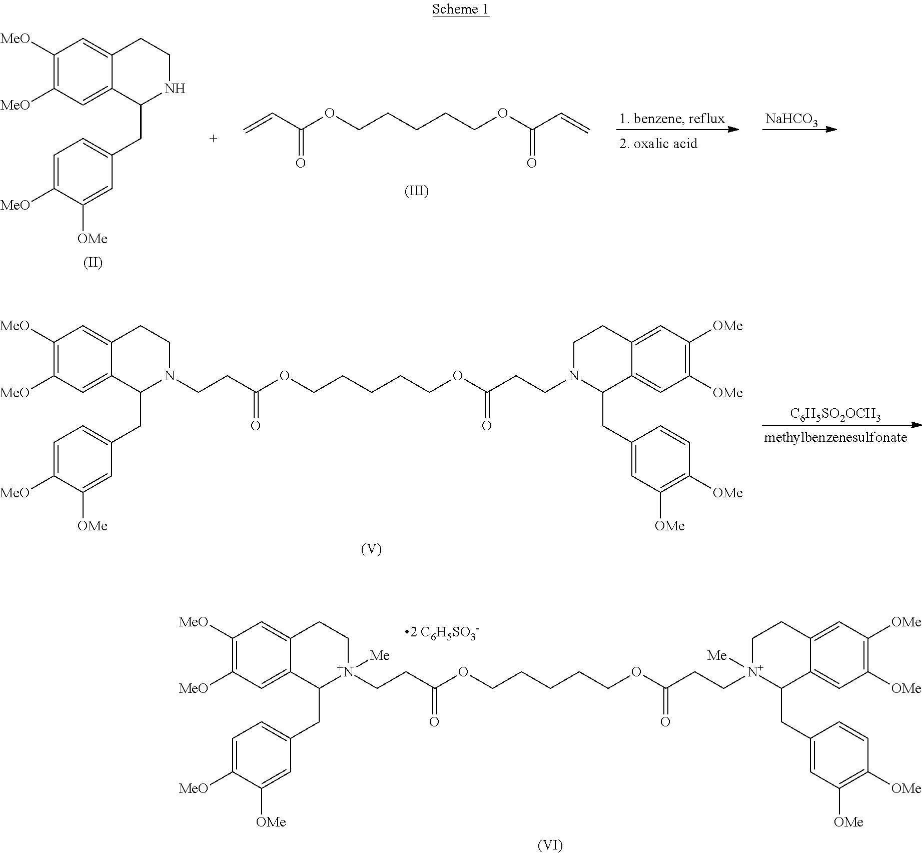

U.S. '507 discloses that the stereoisomerism of atracurium besylate (VI) may be partly controlled by controlling stereochemical configuration of compound (H) to provide the tertiary amine base (V) of a RR-, SS-, or RS-(meso) configuration. The quaternization process introduces 2 additional centers of asymmetry resulting in the formation of a mixture of stereoisomers. U.S. '507 does not describe separating stereoisomers from the mixture.

European application No. 0219616 (hereinafter E.P. '616) discloses the synthesis of atracurium chloride. E.P. '616 describes a process that involves coupling 1-[(3,4-dimethoxyphenyl)methyl]-3,4-dihydro-6,7-dimethoxy-2(1H)-isoquinolinepropanoic acid (compound VII) with 1,5-pentanediol in the presence of an acid to afford the diester (compound IX). The resulting diester is quaternized with methyl iodide to form atracurium iodide, which is then converted into atracurium chloride by means of anion exchange. The process is illustrated in below Scheme 2.

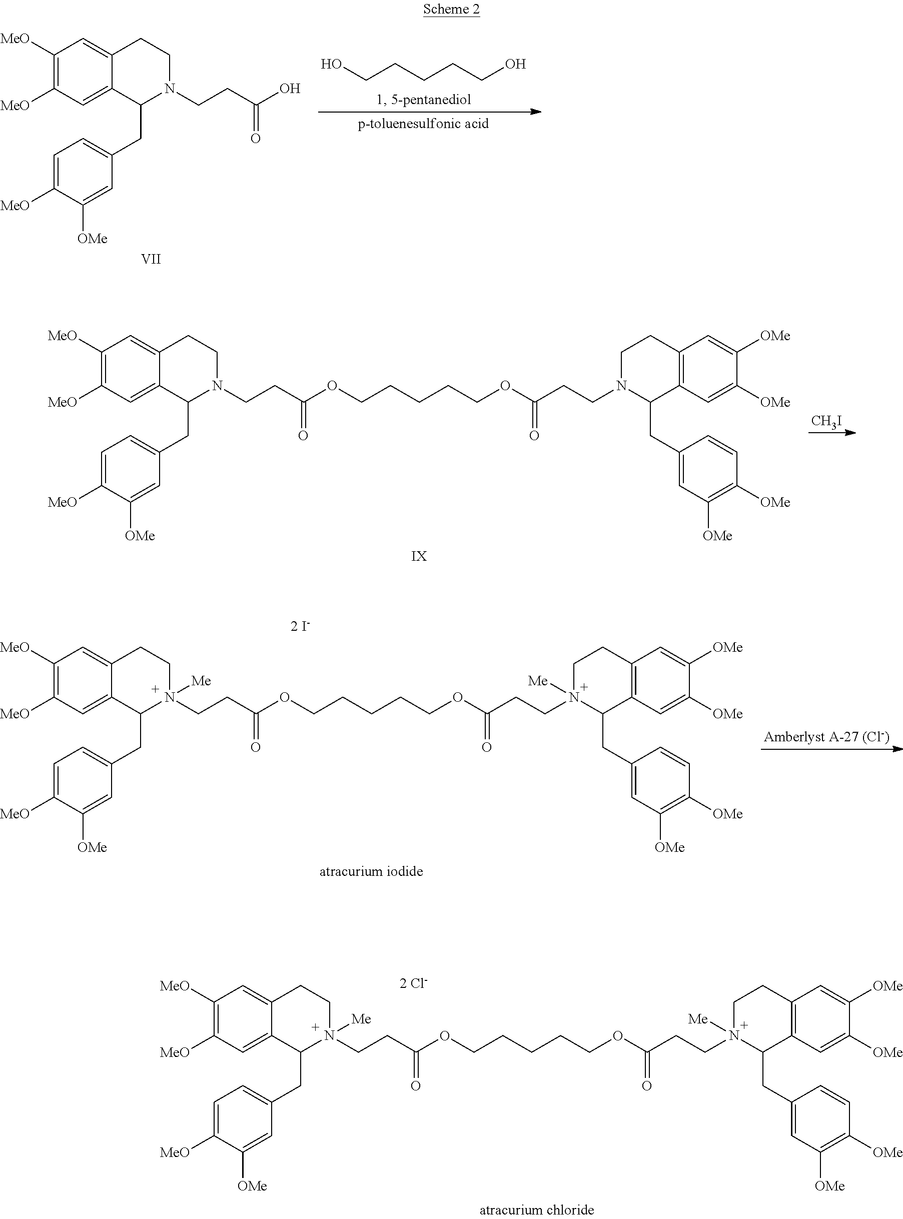

Cisatracurium besylate is disclosed in U.S. Pat. No. 5,453,510 (hereinafter U.S. '510). U.S. '510 describes the formation of (R)-tetrahydropapaverine (compound IIA) from compound (II) which is converted into a mixture of R and S diastereoisomer salts with the chiral amino acid, N-acetyl-L-leucine, resulting in the formation of a mixture of 83% of the R and 17% of the S diastereoisomer. Crystallization of the mixture from acetone affords 97% (R)-tetrahydropapaverine-N-acetyl-L-leucinate and 3% (S)-tetrahydropapaverine-N-acetyl-L-leucinate, which is converted into (R)-tetrahydro-papaverine base. The (R)-tetrahydropapaverine is subsequently reacted with 1,5-pentamethylene diacrylate followed by oxalic acid to afford the dioxalate salt of (1R,1′R)-2,2′-(3,11-dioxo-4,10-dioxamidecamethylene)-bis-(1,2,3,4-tetrahydro-6,7-dimethoxy-1-veratrylisoquinoline) (i.e., an isomer of compound TV). Conversion of the dioxalate salt into the free base, followed by treatment with methyl benzenesulfonate, affords an aqueous solution of (1R,1′R)-atracurium besylate. Lyophilization results in a pale yellow solid that includes a mixture of three isomers, namely, 1R-cis,1′R-cis; 1R-cis,1′R-trans; 1R-trans,1′R-trans (hereinafter referred to as the “atracurium besylate mixture”) in a ratio of about 58:34:6 respectively. The atracurium besylate mixture is subjected to preparative HPLC column chromatography on silica using a mixture of dichloromethane, methanol and benzenesulfonic acid in the ratio of 4000:500:0.25 as the eluent. The fractions containing the required isomer are collected and washed with water. The dichloromethane solution is evaporated to dryness, the residue dissolved in water and the pH of the solution adjusted to 3.5-4.0 with an aqueous solution of benzenesulfonic acid. The aqueous solution is lyophilized to afford cisatracurium besylate possessing an isomeric purity of about 99%.

The drug monograph of atracurium besylate recites 3 impurities, wherein each impurity consists of a mixture of diastereomers. It is well known to skilled artisans that diastereomers are compounds having different chemical and physical characteristics including their molar extinction coefficient (molar absorptivity). The molar extinction coefficient is a measure of light absorbance of a compound at a given wavelength, which is an intrinsic property of the compound. The molar extinction coefficient is dependent on the chemical structure, e.g., the number of aromatic rings, double bonds, etc.

There is a need in the art for compounds and methods for testing the purity of cisatracurium or a salt thereof, e.g., the besylate salt, such as a method of assaying a sample of cisatracurium or a salt thereof, e.g., the besylate salt, for the presence of individual cisatracurium isomers. The present invention provides such compounds and methods.

### BRIEF SUMMARY OF THE INVENTION

The present invention provides single isoquinolinium isomers that can be used as reference markers for the analysis of cisatracurium.

The present invention provides a method of testing the purity of a sample of cisatracurium besylate, which method comprises assaying the sample to detect the presence of at least one of the following compounds, which, according to the present invention, can be used as reference markers: Compound XI, Compound XII, Compound XIII, Compound XVI—the (1R-cis,1′R-trans) isomer of cisatracurium, and Compound XVII—the (1R-trans,1′R-trans) isomer of cisatracurium.

The present invention also provides a process for preparing compounds XI, XII and XIII, which includes reacting the compound (1R-cis)-1-[(3,4-dimethoxyphenyl)-methyl]-1,2,3,4-tetrahydro-6,7-dimethoxy-2-methyl-2-carboxylethyl-isoquinolinium besylate, compound X with the corresponding diol selected from 3-methyl-1,5-pentanediol, 1,5-hexanediol and 1,6-hexanediol.

According to one embodiment of the present invention, the reaction of compound X with the diol is carried out in an organic solvent.

According to another embodiment of the present invention, the reaction of compound X with the dial is optionally carried out in presence of a catalyst.

The present invention further provides Compound XVI—the (1R-cis,1′R-trans) isomer of cisatracurium besylate, which can be produced by reacting cis-(R)-1-[(3,4-dimethoxyphenyl)methyl]-1,2,3,4-tetrahydro-2-[3-[(5-hydroxypentyl)oxy]-3-oxopropyl]-6,7-dimethoxy-2-methyl-isoquinolinium besylate, compound (XIV),

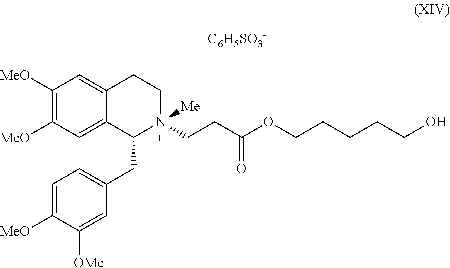

with trans-(R)-1-[(3,4-dimethoxyphenyl)methyl]-1,2,3,4-tetrahydro-6,7-dimethoxy-2-methyl-2-carboxylethyl-isoquinolinium besylate (Compound XV)

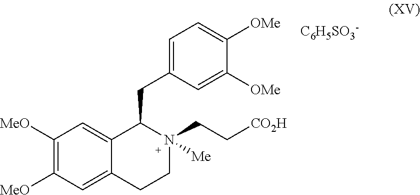

to obtain the (1R-cis,1′R-trans) isomer of cisatracurium besylate and optionally purifying the cisatracurium besylate isomer.

The present invention further provides a Compound XVII—the (1R-trans,1′R-trans) isomer of cisatracurium besylate, which can be produced by reacting Compound (XV) with 1,5-pentanediol in an organic solvent and in the presence of benzenesulfonic acid and optionally purifying the cisatracurium besylate isomer.

The present invention additionally provides a method of testing a sample of cisatracurium salt, e.g., cisatracurium besylate, which includes the steps of:

(a) dissolving a sample of cisatracurium besylate in a solvent to produce a standard solution;

(b) dissolving a sample of the reference marker in a solvent to produce a standard solution of the reference marker;

(c) obtaining the corresponding HPLC chromatograms of the samples prepared in steps (a) and (b); and

(d) calculating the percentage of the reference marker in the tested sample based on the HPLC chromatograms.

### BRIEF DESCRIPTION OF THE DRAWINGS

FIG. 1 illustrates the ¹H-NMR spectrum of Compound XI.

FIG. 2 illustrates the ¹³C-NMR spectrum of Compound XI.

FIG. 3 illustrates the MS spectrum of Compound XI.

FIG. 4 illustrates the ¹H-NMR spectrum of Compound XII.

FIG. 5 illustrates the ¹³C-NMR spectrum of Compound XII.

FIG. 6 illustrates the MS spectrum of Compound XII.

FIG. 7 illustrates the ¹H-NMR spectrum of Compound XIII.

FIG. 8 illustrates the ¹³C-NMR spectrum of Compound XIII.

FIG. 9 illustrates the MS spectrum of Compound XIII.

FIG. 10 illustrates the HPLC chromatogram of a sample containing, inter alia, cisatracurium besylate and at least one reference marker, according to Example 1.

### DETAILED DESCRIPTION OF THE INVENTION

The present invention provides single isoquinolinium compounds that can be used as reference markers for testing the purity of cisatracurium.

The term “reference marker,” as used herein, refers to a compound that can be used for analyzing the purity of an active pharmaceutical ingredient (API) in a sample containing both the API and the reference marker. The analysis can be carried out, e.g., by means of chromatography, e.g., using High Pressure Liquid Chromatography (HPLC).

Applicant has developed a process for preparing cisatracurium besylate, which is depicted in Scheme 3 below, using 1,5-pentanediol as starting material. The process comprises reacting (1R-cis)-1-[(3,4-dimethoxyphenyl)methyl]-1,2,3,4-tetrahydro-6,7-dimethoxy-2-methyl-2-carboxylethyl-isoquinolinium besylate (Compound X) with 1,5-pentanediol optionally in the presence of a catalyst e.g., CaSO₄/benzenesulfonic acid in an organic solvent (e.g., dichloromethane), to form the cisatracurium salt, e.g., cisatracurium besylate.

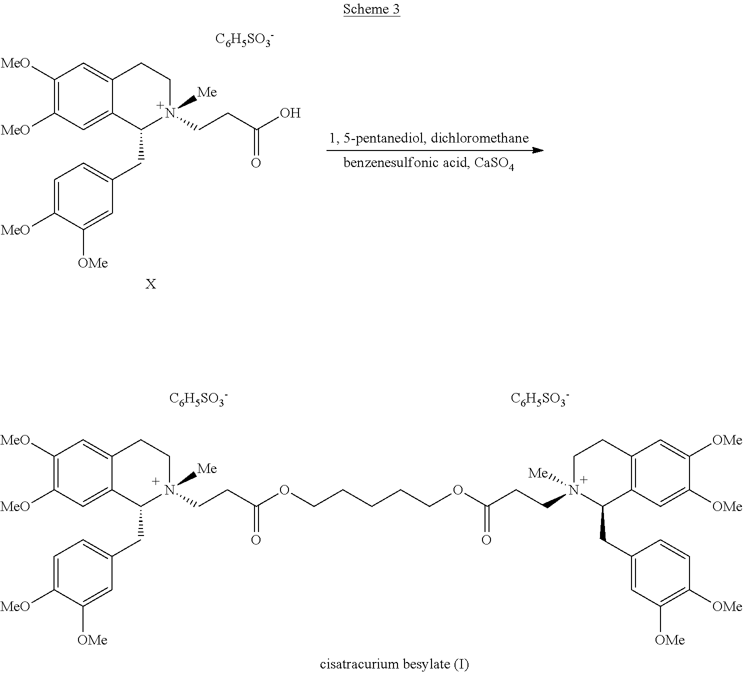

The diol starting material 1,5-pentanediol, used in the preparation of cisatracurium besylate, is often contaminated with structural isomers and homologues, which are very difficult to remove. While checking the commercial 1,5-pentanediol products that are available in the market it turned out that most of them contain at least one of the following impurities: 3-methyl-1,5-pentanediol, 1,6-hexanediol or 1,5-hexanediol. While using 1,5-pentanediol as starting material, the presence of at least one of these diols in the starting material will lead to the formation of several known impurities in the final product, which have similar (but not identical) structure to cisatracurium besylate. Compound XI is derived from 3-methyl-1,5-pentanediol, Compound XII is derived from 1,5-hexanediol and Compound XIII is derived from 1,6-hexanediol. Scheme 4 below depicts the reactions which lead to the formation of the un-wanted impurities which are formed from 3-methyl-1,5-pentanediol, 1,5-hexanediol and 1,6-hexanediol respectively.

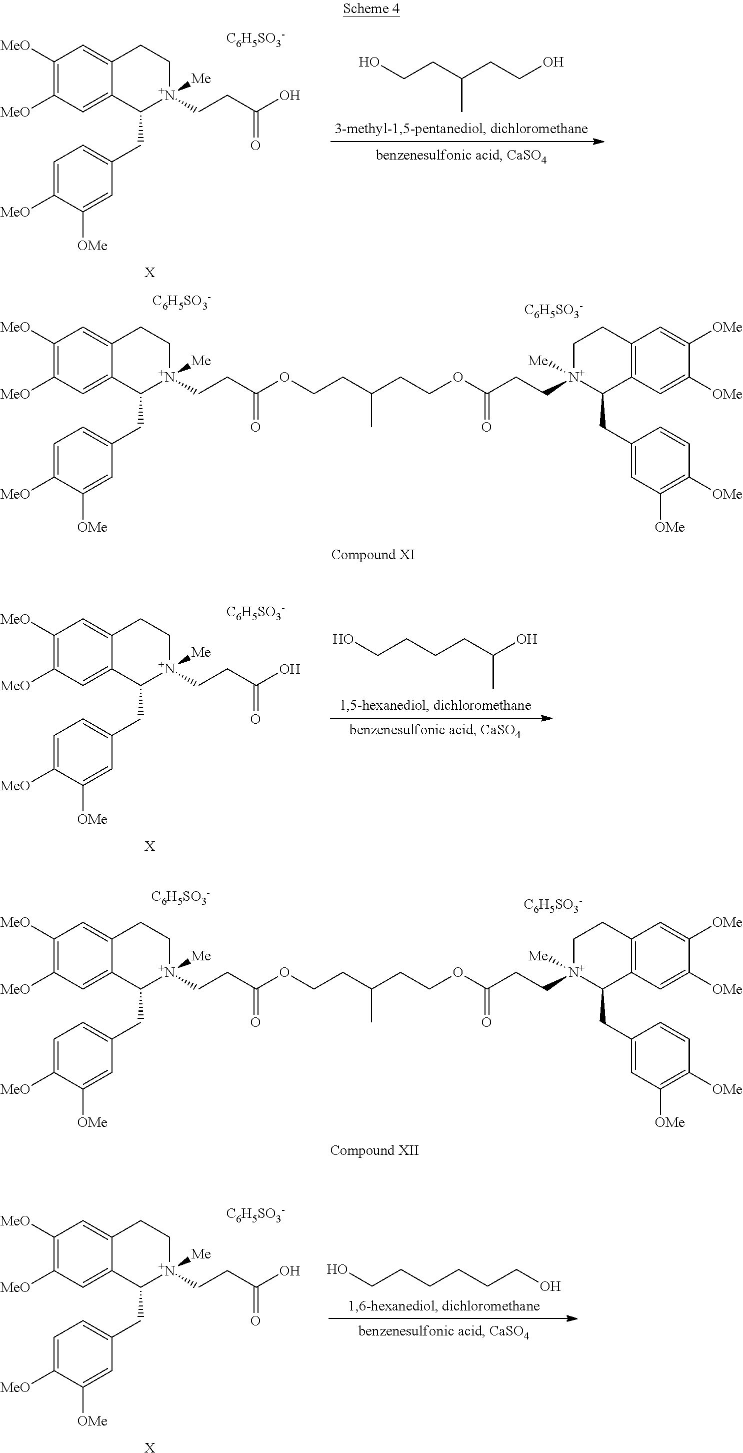

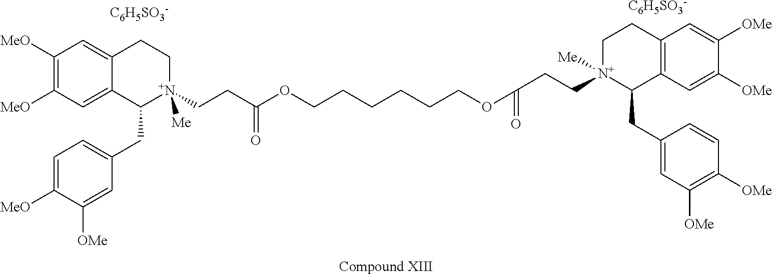

In addition to Compounds XI and XII, two other un-wanted impurities may be formed during the synthesis of cisatracurium besylate, that is, Compound XVI—the (1R-cis,1′S-trans) isomer, and Compound XVII—the (1R-trans,1′R-trans) isomer of cisatracurium besylate. Furthermore, a test sample of the reaction mixture, containing the product cisatracurium besylate, can include other side products such as Compound XVIII-(R)-laudanosine:

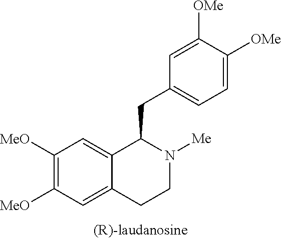

Thus, in one embodiment, the present invention provides a method of testing the purity of a sample of cisatracurium besylate, which method preferably includes assaying the sample to detect the presence of at least one of the following compounds, which, according to the present invention, can be used as reference markers: Compound XI, Compound XII, Compound XIII, Compound XVI—the (1R-cis,1′R-trans) isomer, and Compound XVII—the (1R-trans,1′R-trans) isomer.

The present invention also provides a process for preparing compounds XI, XII and XIII, which includes reacting the compound (1R-cis)-1-[(3,4-dimethoxyphenyl)-methyl]-1,2,3,4-tetrahydro-6,7-dimethoxy-2-methyl-2-carboxylethyl-isoquinolinium besylate, compound X, with the corresponding diol selected from 3-methyl-1,5-pentanediol, 1,6-hexanediol and 1,5-hexanediol.

According to another embodiment of the present invention, the reaction of compound X with the diol is carried out in an organic solvent.

The organic solvent used in the reaction can include, e.g., toluene, one or more xylenes, ethyl acetate, dichloromethane, chloroform or a mixture thereof. A preferred organic solvent is dichloromethane.

According to another embodiment of the present invention, the reaction of compound X with the diol is optionally carried out in presence of a catalyst.

Suitable catalysts include acidic catalysts such as CaSO₄/benzenesulfonic acid, NaHSO₄.SiO₂, Amberlyst® 15 (a sulfonic acid based on crosslinked styrene-divinylbenzene copolymers), and mixtures of benzenesulfonic acid and silica gel, preferably having a pH of from 1.0-4.0. NaHSO₄.SiO₂ is a heterogeneous acidic catalyst that includes sodium hydrogen sulfate supported on silica gel. A preferred acidic catalyst is CaSO₄/benzenesulfonic acid.

The present invention further provides Compound XVI—the (1R-cis,1′R-trans) isomer of cisatracurium besylate, which can be prepared by reacting (R)-1-[(3,4-dimethoxyphenyl)methyl]-1,2,3,4-tetrahydro-2-[3-[(5-hydroxypentyl)oxy]-3-oxopropyl]-6,7-dimethoxy-2-methyl-isoquinolinium besylate compound (XIV)

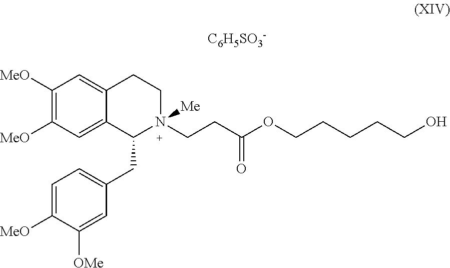

with trans-(R)-1-[(3,4-dimethoxyphenyl)methyl]-1,2,3,4-tetrahydro-6,7-dimethoxy-2-methyl-2-carboxylethyl-isoquinolinium besylate (Compound XV)

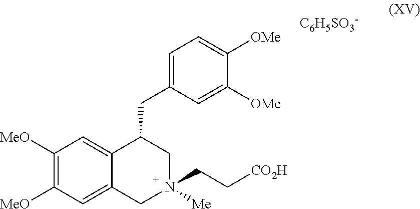

in an organic solvent and in presence of a catalyst to obtain the (1R-cis,1′R-trans) isomer of cisatracurium besylate and optionally purifying the cisatracurium besylate isomer.

The preparation of Compounds XIV and XV is detailed in the experimental section of the present invention.

The organic solvent used in the reaction can include, e.g., toluene, one or more xylenes, ethyl acetate, dichloromethane, chloroform or a mixture thereof. A preferred organic solvent is dichloromethane.

Suitable catalysts include acidic catalysts such as, e.g., CaSO₄/benzenesulfonic acid, NaHSO₄. SiO₂, Amberlyst®15 and mixtures of benzenesulfonic acid and silica gel, preferably having a pH of from 1.0-4.0. NaHSO₄.SiO₂ is a heterogeneous acidic catalyst that includes sodium hydrogen sulfate supported on silica gel. A preferred acidic catalyst is CaSO₄/benzenesulfonic acid.

The present invention further provides Compound XVII—the (1R-trans,1′R-trans) isomer of cisatracurium besylate, which can be prepared by reacting Compound (XV) with 1,5-pentanediol in an organic solvent and in the presence of a catalyst and optionally purifying the cisatracurium besylate isomer.

The organic solvent used in the reaction preferably includes dichloromethane, chloroform, 1,2-dichloroethane, toluene, one or more xylenes, and mixtures thereof. A particularly preferred solvent is dichloromethane.

Suitable catalysts include acidic catalysts such as, e.g., CaSO₄/benzenesulfonic acid, NaHSO₄.SiO₂, Amberlyst®15, and mixtures of benzenesulfonic acid and silica gel, preferably having a pH of from 1.0-4.0. NaHSO₄.SiO₂ is a heterogeneous acidic catalyst that includes sodium hydrogen sulfate supported on silica gel. A preferred acidic catalyst is CaSO₄/benzenesulfonic acid.

As detailed herein, several structural isomers and homologues may be formed during the synthetic course of preparing a cisatracurium salt, e.g., cisatracurium besylate. In accordance with the present invention, such structural isomers and homologues have utility as reference markers for analyzing the purity of cisatracurium besylate, particularly samples that contain such compounds as potential contaminants stemming from side reactions which occur during preparation.

Thus, according to another embodiment, the present invention provides a method of testing the purity of a sample of cisatracurium salt, e.g., cisatracurium besylate, which includes the steps of:

(a) dissolving a sample of cisatracurium besylate in a solvent to produce a standard solution;

(b) dissolving a sample of the reference marker in a solvent to produce a standard solution of the reference marker;

(c) obtaining the corresponding HPLC chromatograms of the samples prepared in steps (a) and (b); and

(d) calculating the percentage of the reference marker in the tested sample based on the HPLC chromatogram.

The test sample, e.g., may be withdrawn from a reaction mixture, which contains the final product, that is, the (1R-cis,1′R-cis) isomer of cisatracurium besylate and at least one impurity corresponding to a reference marker.

The calculation of step (d) can be carried out using the following formula:

$$\% of the reference marker = \frac{{A}_{sample} \times {C}_{std}}{{A}_{std} \times {C}_{sample}} \times P$$

C_{std}=concentration of cisatracurium in the standard solution, mg/mL
Cₛₐₘₚₗₑ=concentration of the test sample, mg/mL
Aₛₐₘₚₗₑ=area of the reference marker in the chromatogram of the test sample
A_{std}=area of cisatracurium in the chromatogram of standard solution
P=purity of cisatracurium in the standard solution (%).

The specific area of the reference marker in the chromatogram of the test sample can be used to calculate the percentage of the reference marker in the tested sample, which is correlated both to the concentration of cisatracurium in the standard solution and the concentration of the test sample.

Reference is now made to the following examples, which, together with the above description, serve to illustrate the invention without limiting its scope. Additional objects, advantages, and novel features of the present invention will become apparent to one ordinarily skilled in the art.

### Example 1

This example details the HPLC method for testing the purity of a sample of cisatracurium besylate by using reference markers.

The columns used were: YMC J′Spher ODS M80, 4.6*250 mm, 4μ, or Inertsil ODS-3, 4.6*250 mm, 5μ, of GL Sciences.

The buffer was prepared by dissolving 5.44 g of KH₂PO₄ in 1000 mL of water (40 mM/L) and the pH was adjusted to 2.1 with phosphoric acid.
Table 1 below details the gradient of the mobile phase which was used, consisting of two eluents:

Eluent A: A mixture of 75% buffer+20% acetonitrile+5% methanol

Eluent B: A mixture of 50% buffer+20% acetonitrile+30% methanol

TABLE 1

| Time (min) | % Eluent A | % Eluent B |
|---|---|---|
| 0 | 80 | 20 |
| 5 | 80 | 20 |
| 15 | 40 | 60 |
| 25 | 40 | 60 |
| 30 | 0 | 100 |
| 44.5 | 0 | 100 |
| 45 | 80 | 20 |

Equilibration time: 15 min
Flow rate: 1.0 mL/min
Column temperature: 40° C.
Detection: 230 nm
Run time: 45 min.
Injection volume: 5 μL
   Diluent: a pH 3 aqueous acidic solution (pH adjusted with phosphoric acid).
   The blank solution was prepared by transferring 0.5 ml of acetonitrile into a 5 mL volumetric flask and completing the volume up to the sign with the diluent under mixing. The tested sample was prepared by weighing 100 mg of the sample into a 20.0 mL volumetric flask and adding 2 ml of acetonitrile under mixing. The volume was completed up to the sign with the diluent under mixing. The diluted solution of cisatracurium reference sample was prepared by weighing 100 mg of cisatracurium reference sample into a 20.0 mL volumetric flask. The volume of the flask was completed with Eluent A under mixing. 1 ml of the thus made solution was transferred into a 20 ml volumetric flask, and the volume was completed with Eluent A. 1 ml of this solution was transferred into a 20 ml volumetric flask and the volume was completed with Eluent A.

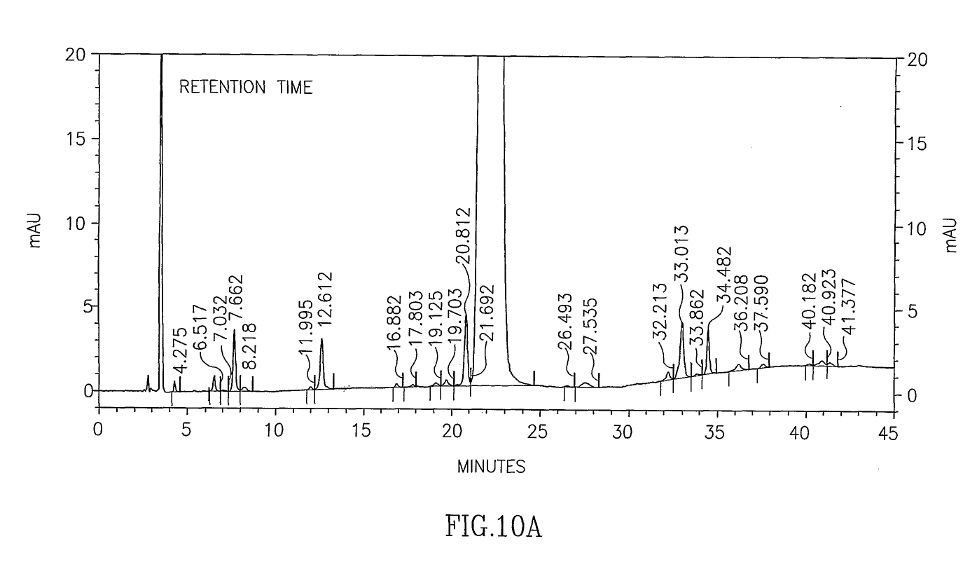

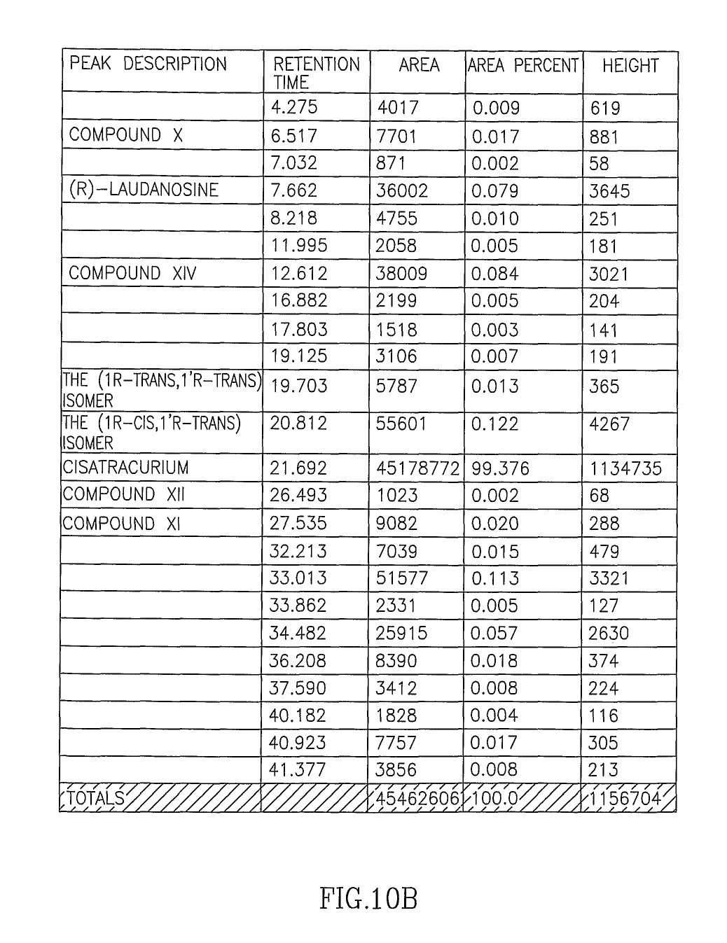

The HPLC chromatogram of a sample containing, inter alia, cisatracurium besylate and at least one reference marker is illustrated in FIG. 10.

### Example 2

This example describes the preparation of Compound XI.

A reaction vessel, equipped with mechanical stirrer and thermometer, was charged under stirring with the 3-methyl-1,5-pentanediol (0.484 g, 0.0041 moles), CaSO₄ (19.8 g) and dichloromethane (33 ml). Stirring was continued for 5 minutes and (1R-cis)-1-[(3,4-dimethoxyphenyl)methyl]-1,2,3,4-tetrahydro-6,7-dimethoxy-2-methyl-2-carboxylethyl-isoquinolinium besylate, Compound (X), was added (5.0 g, 0.0085 moles) and stirring was maintained at 25° C. for 24 hours. A sample was withdrawn and injected to the HPLC system for determining the reaction completion. (If the content of Compound (II) is more than 10%, another portion of CaSO₄ should be added (2.8 g) and stirring should be maintained for additional period of at 25° C. for 24 hours). Then, the reaction mixture was filtered through a Buchner funnel under vacuum to remove the solid CaSO₄ and washed with dichloromethane (10 ml).

The filtrate was washed seven times with water (33 ml each wash) to remove the water-soluble by-product and the layers were separated. The dichloromethane layer was dried over MgSO₄ (3 g) and the solid MgSO₄ was filtered off. The dichloromethane was evaporated to dryness to obtain 2.33 g of Compound XI in 45% yield, having purity of 98.75% (according to HPLC).

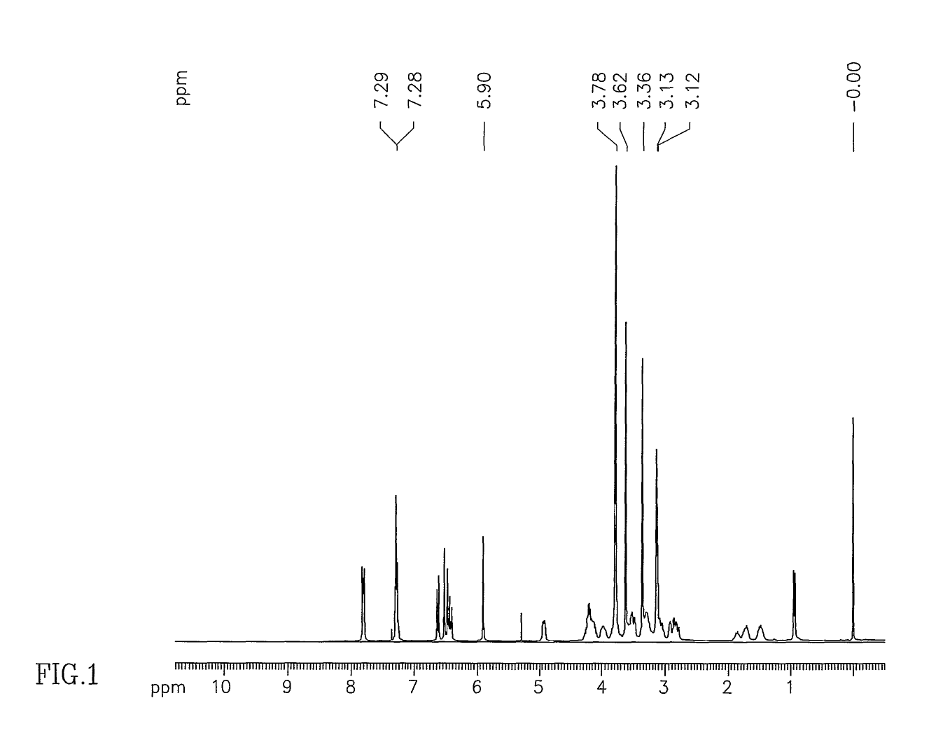

The ¹H-NMR spectrum of Compound XI is illustrated in FIG. 1.

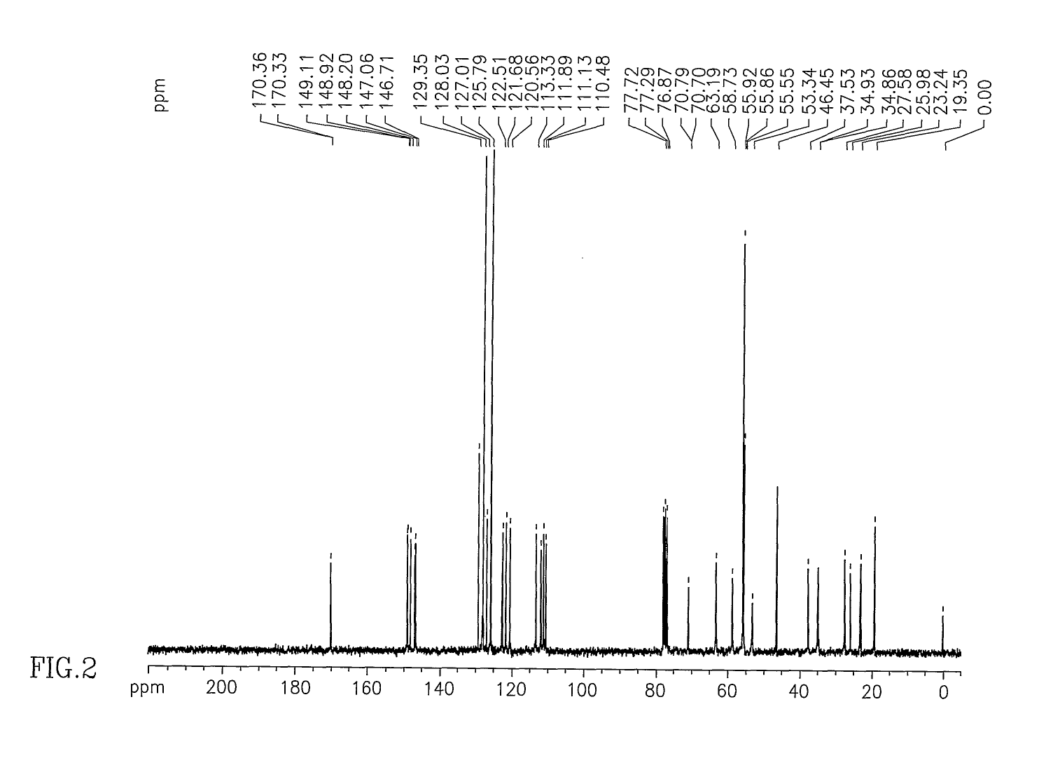

The ¹³C-NMR spectrum of Compound XI is illustrated in FIG. 2.

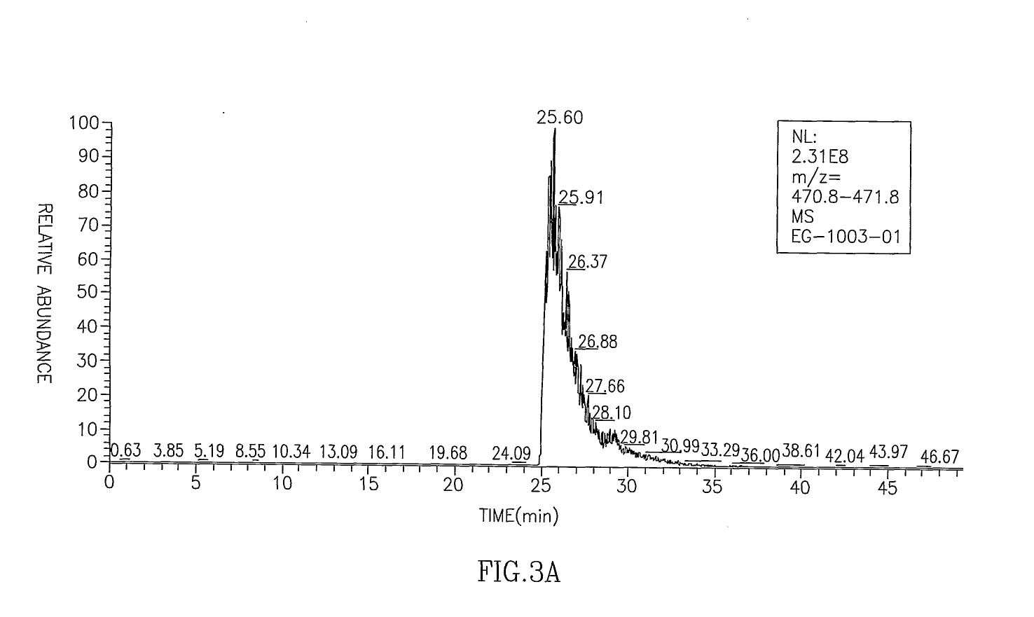

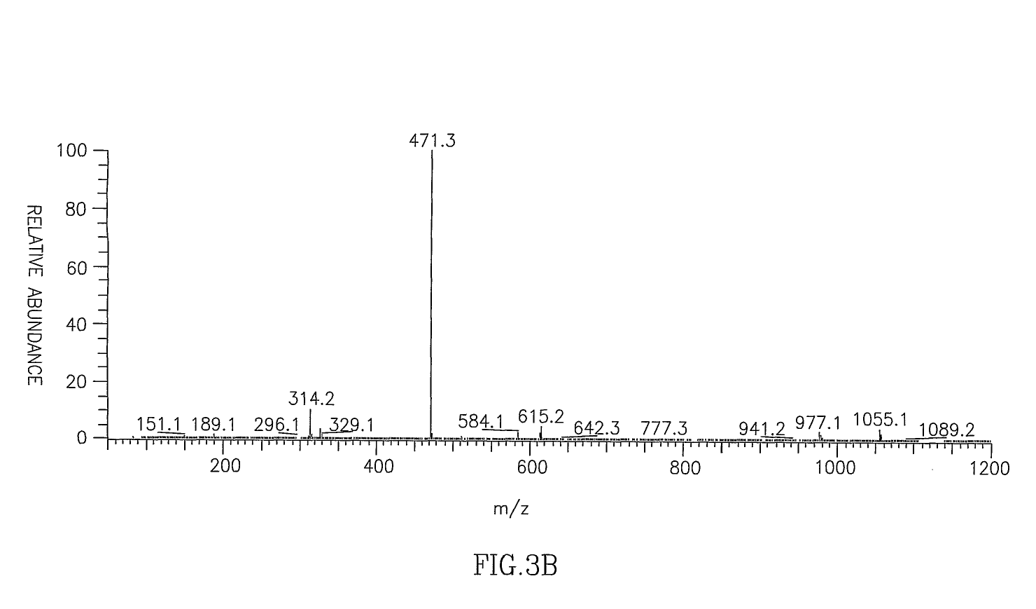

The MS spectrum of Compound XI is illustrated in FIG. 3. The molecular weight of Compound XI is 942.6 g/mole, however since the compound is charged twice, the observed m/z is 942.6/2=471.3.

### Example 3

This example describes the preparation of Compound XII.

A reaction vessel, equipped with mechanical stirrer and thermometer, was charged under stirring with the 1,5-hexanediol (0.484 g, 0.0041 moles), CaSO₄ (19.8 g) and dichloromethane (33 ml). Stirring was continued for 5 minutes and Compound (X) was added (5.0 g, 0.0085 moles) and stirring was maintained at 25° C. for 24 hours. A sample was withdrawn and injected to the HPLC for determining reaction completion. (If the content of Compound (II) is more than 10%, another portion of CaSO₄ should be added (2.8 g) and stirring should be maintained for additional period of at 25° C. for 40 hours). Then, the reaction mixture was filtered through Buchner funnel under vacuum to remove the CaSO₄ and washed with dichloromethane (10 ml).

The filtrate was washed seven times with water (33 ml each wash) to remove the water-soluble by-product and the layers were separated. The dichloromethane layer was dried over MgSO₄ (3 g) and the MgSO₄ was filtered off. The dichloromethane was evaporated to dryness to obtain 1.35 g of Compound XII in 26% yield, having purity of 99.1% (according to HPLC).

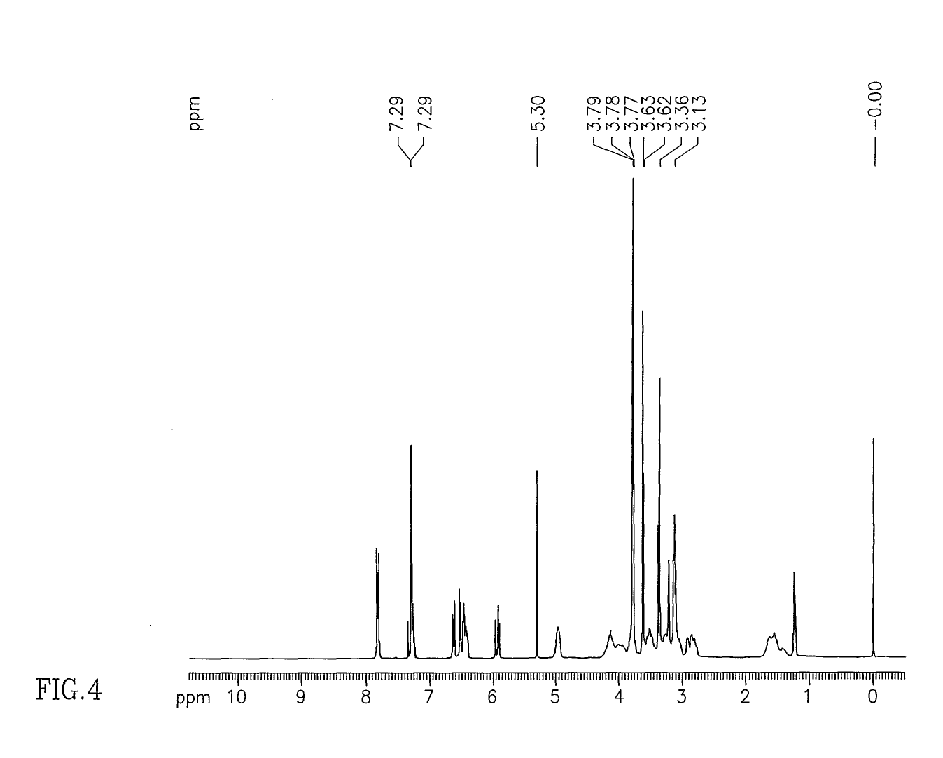

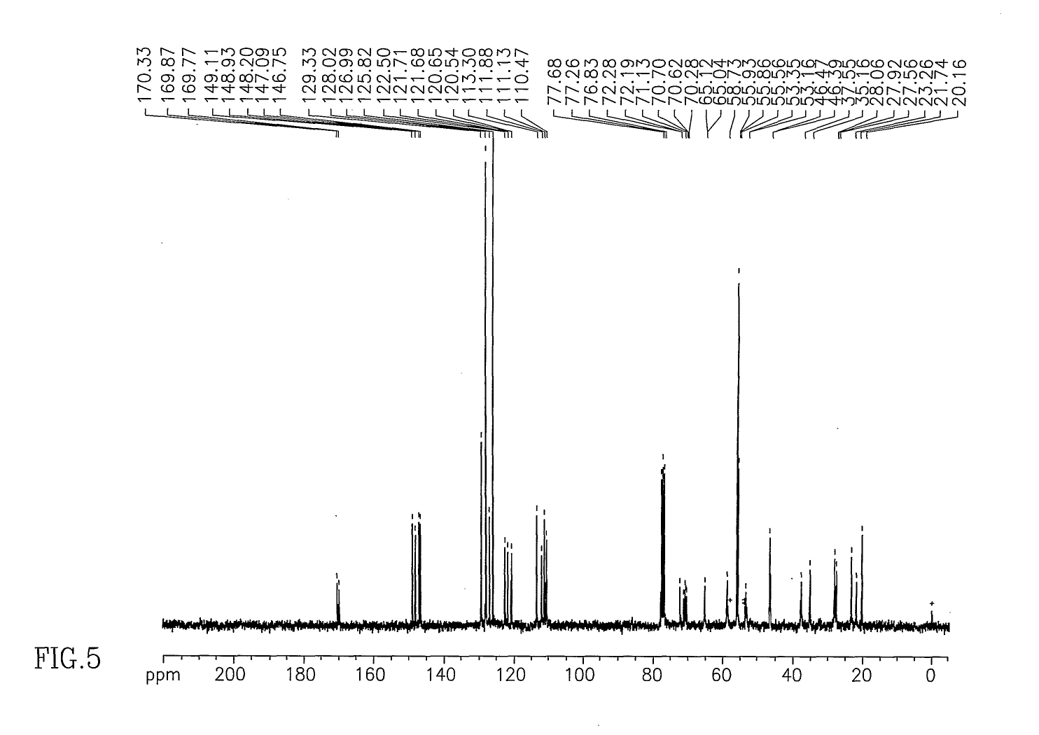

The ¹H-NMR spectrum of Compound XII is illustrated in FIG. 4.

The ¹³C-NMR spectrum of Compound XII is illustrated in FIG. 5.

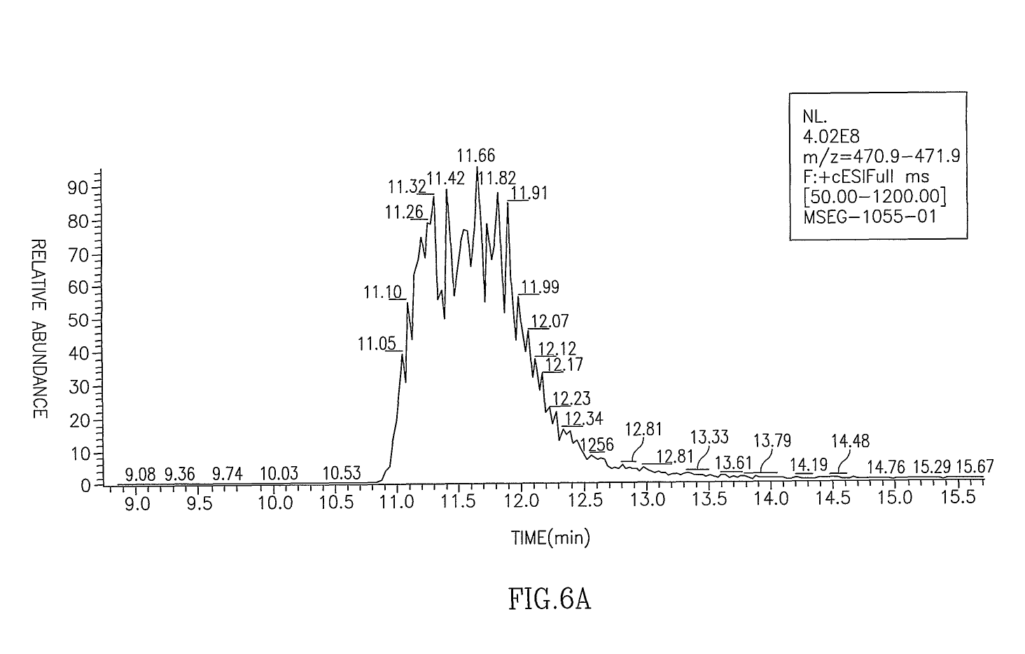

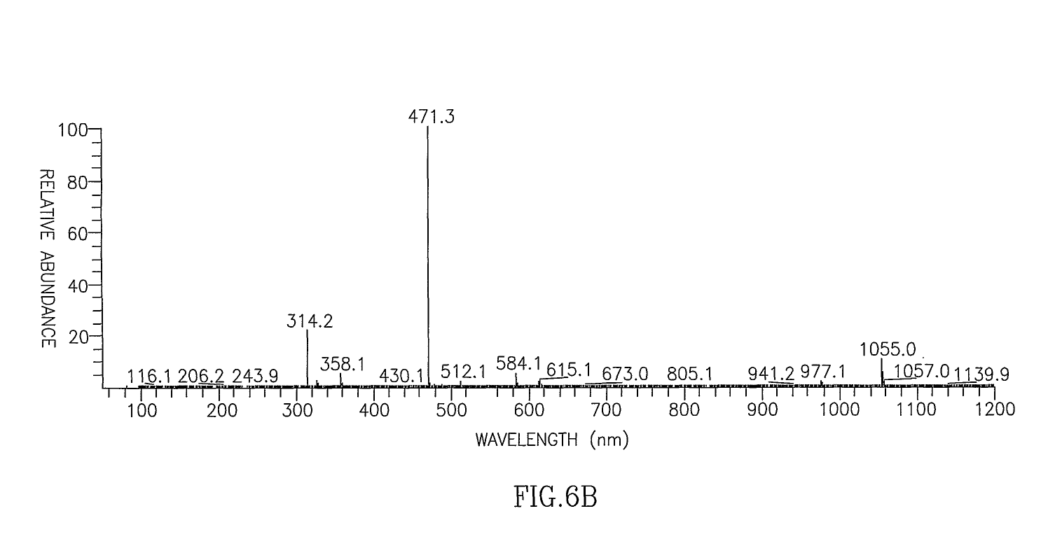

The MS spectrum of Compound XII is illustrated in FIG. 6. The molecular weight of Compound XII is 942.6 g/mole, however since the compound is charged twice, the observed m/z is 942.6/2=471.3.

### Example 4

This example describes the preparation of Compound XIII.

A reaction vessel, equipped with mechanical stirrer and thermometer, was charged under stirring with the 1,6-hexanediol (0.484 g, 0.0041 moles), CaSO₄ (19.8 g) and dichloromethane (33 ml). Stirring was continued for 5 minutes and Compound (X) was added (5.0 g, 0.0085 moles) and stirring was maintained at 25° C. for 24 hours. A sample was withdrawn and injected to the HPLC for determining reaction completion.

(If the content of Compound (X) is more than 10%, another portion of CaSO₄ should be added (2.8 g) and stirring should be maintained for additional period of at ambient temperature for 24 hours). Then, the reaction mixture was filtered through Buchner funnel under vacuum to remove the CaSO₄ and washed with dichloromethane (10 ml). The filtrate was washed seven times with water (33 ml each wash) to remove the water-soluble by-product and the layers were separated. The dichloromethane layer was dried over MgSO₄ (3 g) and the MgSO₄ was filtered off. The dichloromethane was evaporated to dryness to obtain 2.7 g of Compound XIII in 52% yield, having purity of 97.5% (according to HPLC).

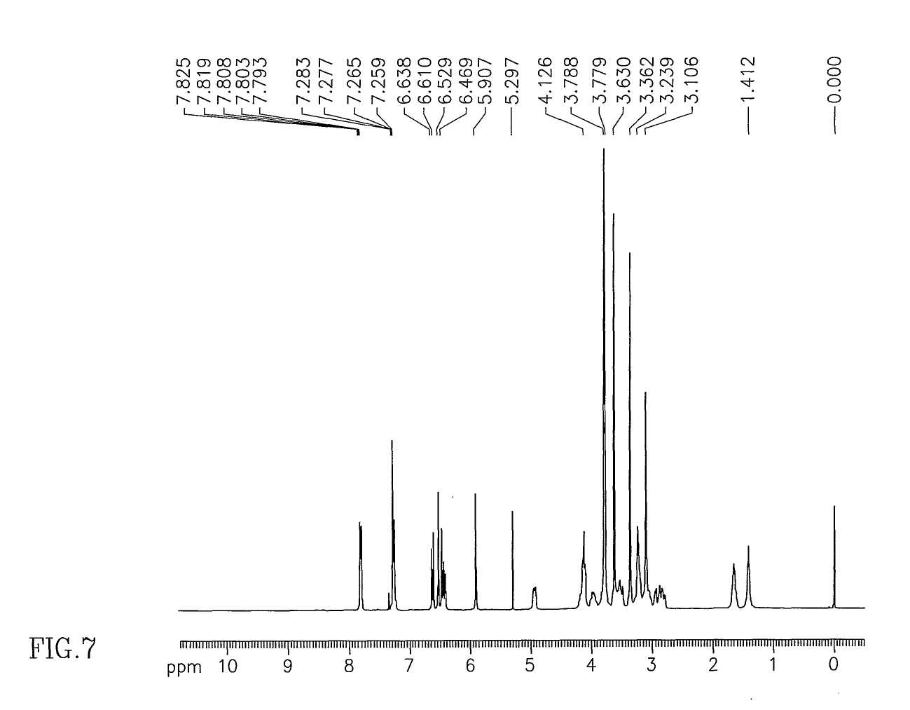

The ¹H-NMR spectrum of Compound XIII is illustrated in FIG. 7.

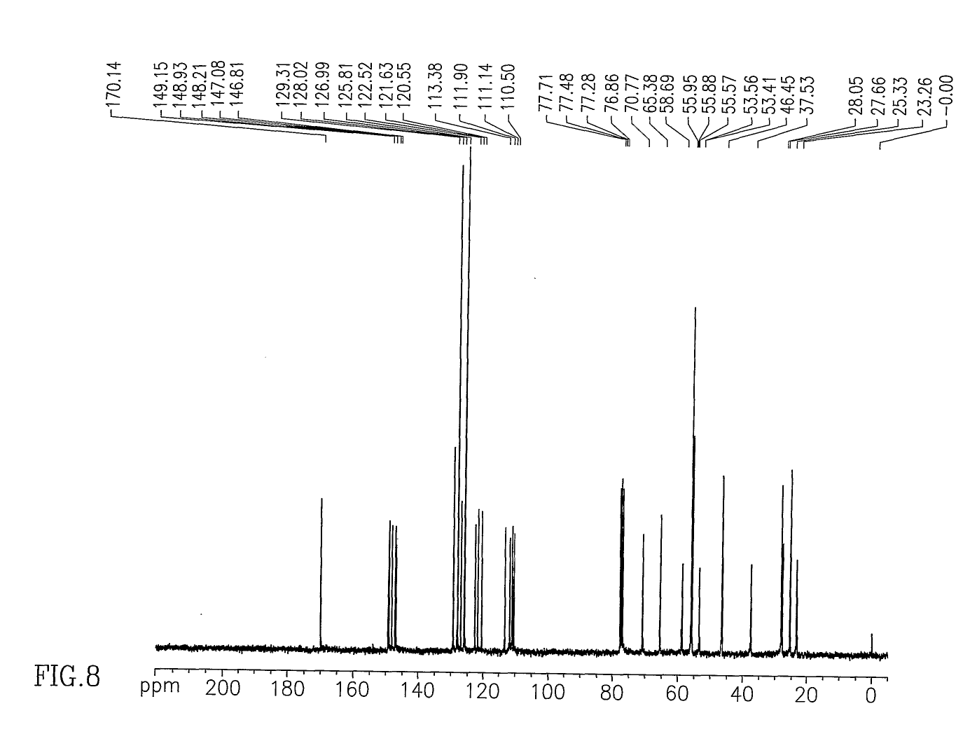

The ¹³C-NMR spectrum of Compound XIII is illustrated in FIG. 8.

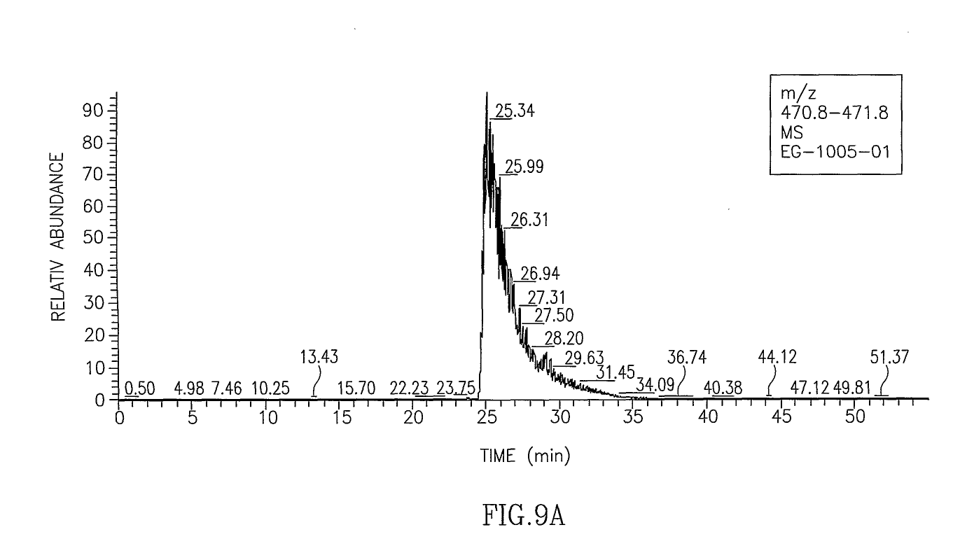

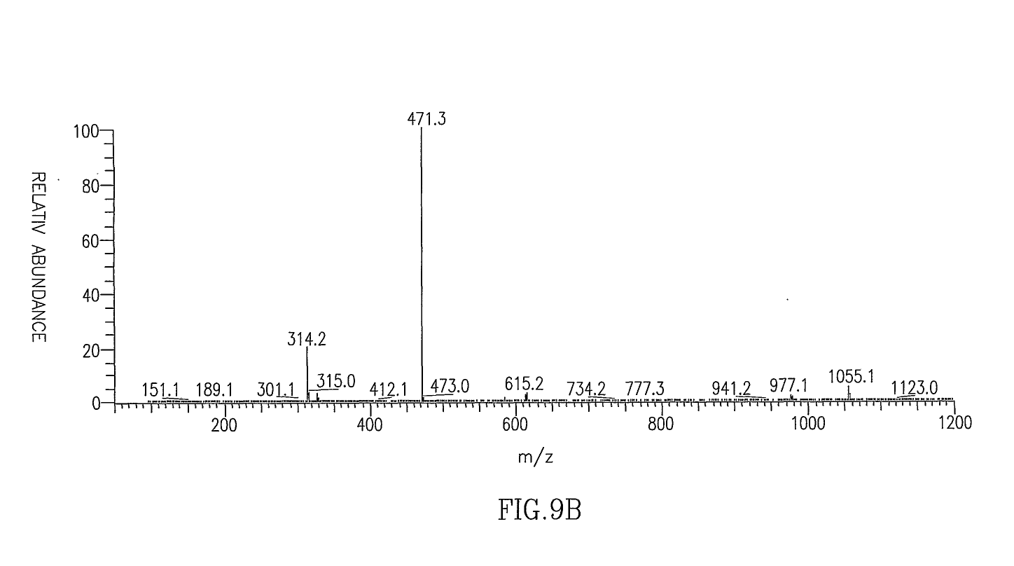

The MS spectrum of Compound XIII is illustrated in FIG. 9. The molecular weight of Compound XIII is 942.6 g/mole, however since the compound is charged twice the observed m/z is 942.6/2=471.3.

### Example 5

This example describes the preparation of (R)—N-(2-tert-butoxycarbonylethyl)-tetrahydropapaverine oxalate.

(R)-Tetrahydropapaverine hydrochloride (30 g, 0.053 moles) was dissolved in water (80 ml) and 25% aqueous ammonium hydroxide solution was added to produce a pH in the range of 9-10. The mixture was extracted with toluene (140 ml) and the organic phase was washed with brine and dried over MgSO₄. The solution was concentrated to 50 ml, tert-butyl acrylate (9.3 ml) and glacial acetic acid (1.6 ml) were added to the solution and the mixture was heated at 80° C. for 5 hours. The mixture was cooled to ambient temperature and a solution of oxalic acid dihydrate (7.4 g, 1.1 eq.) in acetone (35 ml) was added. Ethyl acetate (100 ml) was added to the thus formed suspension and a precipitate was collected by filtration, washed with ethyl acetate and dried at 50° C. overnight to yield (R)—N-(2-tert-butoxycarbonylethyl)-tetrahydropapaverine oxalate (26 g, 88% yield).

### Example 6

This example describes the preparation of pure (R, trans)-N-(2-tert-butoxycarbonylethyl)-N-methyl-tetrahydropapaverinium besylate

(R)—N-(2-tert-butoxycarbonylethyl)-tetrahydropapaverine oxalate (20.0 g, 0.0356 mol) was dissolved in water (200 ml) and 25% aqueous NaOH solution was added to produce pH 10. The mixture was extracted with dichloromethane (3×100 ml) and the organic phase was washed with brine and dried over magnesium sulfate. The solvent was then removed from the solution under reduced pressure to obtain residual oil. Acetonitrile (10 ml) and methyl besylate (9.7 ml, 2.0 eq.) were added to the oil and the mixture was stirred at 30-35° C. for 24 hours (HPLC: 78.34% of cis-isomer and 21.66% of the trans-isomer). Dichloromethane (30 ml) was added to the mixture to obtain a solution. Diethyl ether (50 ml) was added to the solution and the mixture was stirred at ambient temperature overnight. A colorless precipitate was collected by filtration, washed with dichloromethane-diethyl ether mixture (3:4) and dried at ambient temperature in vacuum desiccator for 5 hours to obtain (R, trans)-N-(2-tert-butoxycarbonylethyl)-N-methyl-tetrahydropapaverinium besylate (3.1 g, 15% yield; purity by HPLC: 99.33%; containing 0.67% of cis-isomer). The filtrate contained 93.59% of the cis-isomer and 6.41% of the trans-isomer.

The obtained (R, trans)-N-(2-tert-butoxycarbonylethyl)-N-methyl-tetrahydropapaverinium besylate was treated with dichloromethane:diethyl ether mixture (3:4) under stirring at ambient temperature for 3 hours to obtain pure (R, trans)-N-(2-tert-butoxycarbonylethyl)-N-methyl-tetrahydropapaverinium besylate (purity by HPLC: 99.8%). ¹H NMR (CDCl₃): δ=1.36 (s, 9H, t-butyl), 2.83-4.03 (m, 10H, H₃, H₄, H₁₁, H₁₈, and H₁₉), 3.34 (s, 3H, NMe), 3.60 (s, 3H, OCH₃), 3.68 (s, 3H, OCH₃), 3.77 (s, 3H, OCH₃), 3.82 (s, 3H, OCH₃), 4.74 (m, 1H, H₁), 5.61 (s, 1H, H₈), 6.41 (m, 1H, H₁₇), 6.60 (m, 2H, H₁₃ and H₅), 6.75 (m, 1H, H₁₆), 7.31-7.35 (m, 3H, besylate), 7.27-7.32 (m, 5H, Ph), and 7.89-7.92 (m, 2H, besylate). ¹³C NMR (CDCl₃): δ=23.38 (C₄), 27.92 (C—CH₃), 28.88 (C₁₉), 37.58 (C₁₁), 49.00 (NCH₃), 53.64 (C₃), 55.47 (OCH₃), 55.82 (OCH₃), 55.86 (OCH₃), 56.15 (OCH₃), 56.84 (C₁₈), 70.64 (C₁), 82.83 (CMe₃), 110.65 (C₅), 110.96 (C₁₆), 111.73 (C₈), 113.70 (C₁₃), 120.23 (C₁₀), 121.24 (C₉), 122.96 (C₁₇), 125.92 (CH, besylate), 127.44 (C₁₂), 128.10 and 129.40 (CH, besylate), 146.59 (C, besylate), 146.83 (C₆), 148.07 (C₁₄), 148.97 (C₁₅), 149.17 (C₇), and 168.58 (C₂₀).

### Example 7

This example describes the preparation of (1R,trans)-1-[(3,4-dimethoxy-phenyl)methyl]-1,2,3,4-tetrahydro-6,7-dimethoxy-2-methyl-2-carboxyethyl-isoquinolinium besylate (Compound XV).

A mixture of (R,trans)-N-(2-tert-butoxycarbonylethyl)-N-methyl-tetrahydropapaverinium besylate (2.0 g, 98.5% purity), Amberlyst®15 hydrogen form (0.5 g) and water (10 ml) was stirred at 45-55° C. for 8 hours. Then, the Amberlyst®15 hydrogen form was collected by filtration and the filtrate was filtered off via Celite to obtain a clear solution. The water was removed from the solution under reduced pressure at 30-40° C. to afford (1R, trans)-1-[(3,4-dimethoxyphenyl)methyl]-1,2,3,4-tetrahydro-6,7-dimethoxy-2-methyl-2-carboxylethyl-isoquinolinium besylate as a foam (1.82 g, 100% yield; purity by HPLC: 98.5%). The foam was dissolved in acetone (20 ml) and the solution was stirred at ambient temperature for 2 h to obtain a suspension. A solid was collected by filtration, washed with acetone and dried at 30° C. under reduced pressure overnight to afford a crystalline (1R,cis)-1-[(3,4-dimethoxyphenyl)methyl]-1,2,3,4-tetrahydro-6,7-dimethoxy-2-methyl-2-carboxylethyl-isoquinolinium besylate (1.7 g, 92.9% yield, purity by HPLC: 99.0%); mp 181-184° C.

### Example 8

This example describes the preparation of cis-(R)-1-[(3,4-dimethoxyphenyl)-methyl]-1,2,3,4-tetrahydro-2-[3-[(5-hydroxypentyl)oxy]-3-oxopropyl]-6,7-dimethoxy-2-methyl-isoquinolinium besylate compound (XIV).

1,5-Pentanediol (14.8 g, 136 mmol, 20 eq.) was added to 70 mL of anhydrous methylene chloride. The flask was sealed and placed under argon. Benzenesulfonic acid (1.08 g, 6.8 mmol, 1 eq.) and CaSO₄ (16 g) were added and the suspension was stirred for 5 minutes before cis-(R)—N-(2-carboxylethyl)-N-methyl-tetrahydropapaverinium besylate (4 g, 6.8 mmol) was added. The reaction mixture was stirred at ambient temperature overnight. The suspension was filtered off through a Buchner funnel. Methylene chloride (30 mL) was added to the thus formed solution, which was washed with water (3×40 mL). The organic phase was dried over MgSO₄ and the solvent was removed under reduce pressure to afford a white solid (4.3 g, 6.49 mmol, 95% yield). According to the HPLC analysis, the sample contained 93% of the cis mono ester, 0.5% of cis-(R)—N-(2-hydroxycarbonylethyl)-N-methyl-tetrahydropapaverinium besylate, and 6.5% cisatracurium besylate.

### Example 9

This example describes the preparation of the (1R-cis,1′R-trans) cisatracurium isomer.

Benzenesulfonic acid (269 mg, 1 eq.), CaSO₄ (4 g) and dichloromethane (25 mL) were added to a dry flask. The flask was stirred under argon for 1 minute at ambient temperature. Compound XV (1 g, 1.7 mmol) and Compound XIV (1.146 g, 1.7 mmol, 1 eq.) were added. The thus formed suspension was stirred for the weekend under argon at ambient temperature. Dichloromethane (10 mL) was added and the solid was filtered off through a Buchner funnel. The organic phase was washed with water (2×15 mL). The organic phase was dried over MgSO₄ and the solvent was removed under reduce pressure to afford white solid (1.512 g, 1.22 mmol, 72% yield). The 1R-cis,1′R-trans isomer was purified by means of HPLC separation, which was carried out using a normal phase column (Alltima, Silica, 5μ, 250 mm×22 mm, SN:606061455.1, Lot. No. 0507000057). The Mobile phase was 80% DCM 20% methanol with 0.5% benzenesulfonic acid, isocratic conditions 10 mL/min. The solvent was removed under reduce pressure to give a colorless viscous oil (400 mg, 0.323 mmol, 19% yield, 97% purity).

### Example 10

This example describes the preparation of the (1R-trans,1′R-trans) cisatracurium isomer.

1,5-Pentanediol (45.798 mg, 0.44 mmol, 0.48 eq.) was added to 10 mL of anhydrous dichloromethane. The flask was sealed and placed under argon. Benzenesulfonic acid (144.95 mg, 1 eq.) and CaSO₄ (2 g) were added and the suspension was stirred for 15 minutes before Compound XV (500 mg, 0.9174 mmol) was added. The reaction mixture was stirred at ambient temperature overnight. Dichloromethane (20 mL) was added to the thus formed suspension, which was filtered off through a Buchner funnel. The organic phase was washed with water (3×10 mL), dried over MgSO₄ and the solvent was removed under reduce pressure to afford a white solid (408 mg, 0.33 mmol, 75% yield) containing 97% of the 1R-trans,1′R-trans cisatracurium isomer, as determent by HPLC.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound, in substantially purified form, selected from: 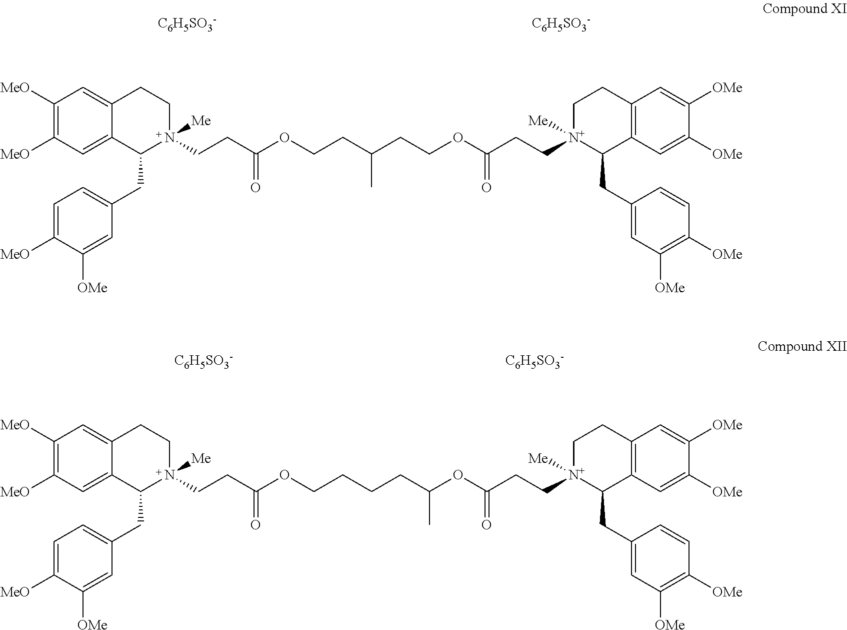 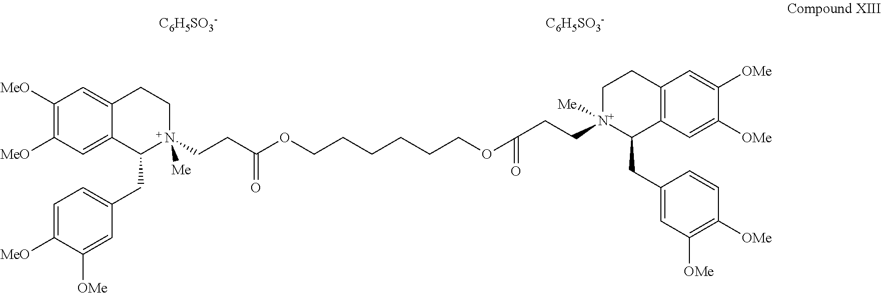
the (1R-cis,1′R-trans) isomer of cisatracurium (Compound XVI), and
the (1R-trans,1′R-trans) isomer of cisatracurium (Compound XVII).

2. The compound of claim 1 having purity which is equal to or greater than 97%.

3. A method of testing the purity of a sample of cisatracurium besylate, which method comprises:
(a) dissolving a sample of cisatracurium besylate in a solvent to produce a standard solution;
(b) dissolving a sample of a reference marker in a solvent to produce a standard solution of the reference marker;
(c) obtaining the corresponding HPLC chromatograms of the samples prepared in steps (a) and (b); and
(d) calculating the percentage of the reference marker in the tested sample based on the HPLC chromatograms.

4. The method of claim 3, wherein the reference marker is selected from at least one of Compound XI, Compound XII, Compound XIII, Compound XVI—the (1R-cis,1′R-trans) isomer, and Compound XVII—the (1R-trans,1′R-trans) isomer.

5. A process for preparing compounds XI, XII or XIII, the process comprising reacting the compound (1R-cis)-1-[(3,4-dimethoxyphenyl)methyl]-1,2,3,4-tetrahydro-6,7-dimethoxy-2-methyl-2-carboxylethyl-isoquinolinium besylate (Compound X) with the corresponding diol selected from 3-methyl-1,5-pentanediol, 1,6-hexanediol and 1,5-hexanediol.

6. The process of claim 5, wherein the reaction is carried out in an organic solvent.

7. The process of claim 6, wherein the organic solvent is toluene, one or more xylenes, ethyl acetate, dichloromethane, chloroform or a mixture thereof.

8. The process of claim 7, wherein the organic solvent is dichloromethane.

9. The process of claim 5, wherein the reaction is carried out in the presence of a catalyst.

10. The process of claim 9, wherein the catalyst is CaSO₄/benzenesulfonic acid, NaHSO₄.SiO₂, AMBERLYST® 15, and mixtures of benzenesulfonic acid and silica gel having a pH of from 1.0-4.
